(19) United States
(12) Patent Application Publication
(10) Pub. No.: US 2020/0197355 A1
(43) Pub. Date: Jun. 25, 2020

(54) METHODS, COMPOSITIONS AND CONTAINERS FOR REDUCING SOLID FORM QUERCETIN DEGRADATION AND 2-(3,4-DIHYDROXYBENZOYLOXY)-4,6-DIHYDROXYBENZOIC ACID TOXIC BYPRODUCTS THEREOF

(71) Applicant: PUBLIC JOINT STOCK COMPANY "SCIENTIFIC INDUSTRIAL CENTRE "BORSHCHAHIVSKIY CHEMICAL-PHARMACEUTICAL PL, Kiev (UA)

(72) Inventors: Sergii Fesenko, Kiev (UA); Alexander Shneider, Dedham, MA (US); Vladimir Gabai, Brighton, MA (US)

(21) Appl. No.: 16/226,781

(22) Filed: Dec. 20, 2018

(51) Int. Cl. A61K 31/352; A61K 9/19; A61K 47/32; A61K 47/02

(52) U.S. Cl. CPC A61K 31/352; A61K 47/02; A61K 47/32; A61K 9/19

(57) ABSTRACT

Provided herein are compositions and methods which reduce degradation of solid form quercetin compositions and reduce the formation of a toxic compound, 2-(3,4-dihydroxybenzoyloxy)-4,6-dihydroxybenzoic acid. Also provided are containers and kits that contain solid form quercetin compositions with reduced degradation of quercetin and reduced formation of 2-(3,4-dihydroxybenzoyloxy)-4,6-dihydroxybenzoic acid. The provided composition and methods increase the shelf life and patient safety of solid form quercetin compositions.

### FIELD OF INVENTION

The invention relates to methods and compositions for the prevention of degradation of quercetin and reduction of the formation of a toxic product 2-(3,4-dihydroxybenzoyloxy)-4,6-dihydroxybenzoic acid (DB-DBA). The invention also includes related storage containers. More specifically, the invention relates to the storage of lyophilized quercetin compositions stored in a non-reactive gas atmosphere at ambient temperature.

### BACKGROUND OF THE INVENTION

Quercetin is a plant flavonoid whose inclusion in human diet has been widely associated with a number of health benefits. These benefits include: 1) antioxidant; 2) anti-inflammatory; 3) antiviral; and 4) anticancer activities (Wang et al., 2016). Quercetin is also used to ease cardiovascular diseases (i.e., heart disease, hypertension, and high blood cholesterol).

The bioavailability of quercetin in humans is low and highly variable (0-50%), and it is rapidly cleared with an elimination half-life of 1-2 hours after ingestion in foods or supplements (Graefe et al., 2001). There are several delivery systems to increase quercetin bioavailability: 1) lipid-based carriers; 2) polymer-based carriers or nanoparticles; 3) inclusion complexes; 4) micelles; and 5) conjugates-based capsulations (Wang et al., 2016). One such polymer-based carrier is polyvinylpyrrolidone (PVP). One PVP-based formulation of quercetin provides a 20,000-fold increase in quercetin solubility (Porcu et al., 2018).

CORVITIN® (PJSC SIC “Borshchahivskiy CPP”, Kiev, Ukraine), which combines quercetin with PVP in solid form, is suitable for intravenous injections when dissolved in saline. Quercetin/PVP formulations lower blood pressure in rats both in short-term and long-term bases (Porcu et al., 2018). Prolonged administration (1 month) of CORVITIN® to rabbits following a cholesterol-rich diet significantly decreased atherosclerotic lesion areas in the aorta (Pashevin et al., 2011). CORVITIN® treatment improves cardiac hemodynamics. CORVITIN® treatment also reduces cardiac fibrosis (Kuzmenko et al., 2013).

CORVITIN® administered to patients with acute myocardial infarction decreases the activity of myeloperoxidase in plasma of blood, which is a marker of the metabolic activity of phagocytes and inflammation(Ryzhkova et al., 2016). CORVITIN® treatment results in decreased blood pressure, pulse pressure, improved structural and functional characteristics of the myocardium (including the increase in ejection fraction (EF), and significant decrease of left ventricular end-diastolic dimension (LVEDd), end-diastolic volume (EDV), left ventricular mass index (LVMI), reduced NT-proBNP levels, total NO and improved heart rate variability (Denina, 2013). CORVITIN® is approved in the Ukraine for therapy in patients suffering myocardial infarction and related diseases.

Lyophilization of drugs, particularly biopharmaceuticals, is often used when a drug ingredient is unstable in liquid or frozen form. In addition, lyophilization allows the storage of material for longer periods of time and at room temperature. No studies on the stability of lyophilized quercetin compositions have been performed previously.

### SUMMARY OF THE INVENTION

Provided herein are formulations including a solid form quercetin composition in an enclosed atmosphere consisting essentially of an inert gas or a combination of inert gases. In some embodiments the solid form quercetin composition is freeze-dried or prepared by spraying drying, rotoevaporation, or crystallization. In some embodiments, the inert gas is essentially oxygen-free. In some embodiments, the inert gas is nitrogen or argon. In some embodiments, the solid form quercetin includes a drug delivery formulation. In some embodiments, the drug delivery formulation includes a lipid-based carrier, a polymer-based carrier, nanoparticles, inclusion complexes, micelles, or a conjugate-based capsulation. In some embodiments, the polymer-based carrier includes polyvinylpyrrolidone (PVP). In some embodiments, the solid form quercetin composition includes about 7-11% quercetin and about 89-93% polyvinylpyrrolidone w/w. In some embodiments, the solid form quercetin composition remains greater than 99% of the input quercetin after 24 months at about 20-25° C. or greater than 99% of the input quercetin after 24 months days at about 21° C., or greater than 97% of the input quercetin after 24 months at about 20-25° C., or greater than 97% of the input quercetin after 24 months at about 21° C.

Provided herein are formulations including a solid form quercetin composition wherein the solid form quercetin composition comprises less than 1% 2-(3,4-dihydroxybenzoyloxy)-4,6-dihydroxybenzoic acid after 24 months at about 20-25° C. or less than about 1% of 2-(3,4-dihydroxybenzoyloxy)-4,6-dihydroxybenzoic acid after 24 months at about 21° C. Also provided are the above formulations comprising less that about 0.5% of 2-(3,4-dihydroxybenzoyloxy)-4,6-dihydroxybenzoic acid. In some embodiments, the solid form quercetin composition after 24 months at 20-25° C. or at 21° C. has a 72 h-EC50 less than 7.5 mg/ml in a cytotoxicity assay with

MDA-MB231 human breast cancer cells. Also provided are the above formulations having a 72 h-EC50 less than 750 microgram/ml in a cytotoxicity assay with MDA-MB231 human breast cancer cells. In some embodiments, the above formulations include an enclosed atmosphere consisting essentially of an inert gas or a combination of inert gases.

Provided herein are methods of reducing the rate of formation of a byproduct of degradation of a solid form quercetin composition by purging air from an airtight container containing the solid form quercetin composition and filling the container with an atmosphere consisting essentially of an inert gas or a combination of inert gases. In some embodiments, the solid form quercetin composition is freeze-dried or prepared by spraying drying, rotoevaporation, or crystallization. In some embodiments, the inert gas is essentially oxygen-free. In some embodiments, the inert gas includes nitrogen or argon. In some embodiments, the solid form quercetin composition includes a drug delivery formulation. In some embodiments, the drug delivery formulation includes a lipid-based carrier, a polymer-based carrier, nanoparticles, inclusion complexes, micelles, or a conjugate-based capsulation. In some embodiments, the polymer-based carrier includes polyvinylpyrrolidone (PVP). In some embodiments, the solid form quercetin composition includes about 8:1, about 9:1, about 10:1, about 11:1 or about 12:1 polyvinylpyrrolidone:quercetin w/w. In some embodiments, the solid form quercetin composition can be 7-11% quercetin and about 89-93% polyvinylpyrrolidone w/w. In some embodiments, the airtight container includes a glass vial with a stopper and aluminum cap or a glass ampoule. Also provided are any of the above methods wherein the byproduct includes 2-(3,4-dihydroxybenzoyloxy)-4,6-dihydroxybenzoic acid.

Provided herein are containers including any of the above solid form quercetin compositions and an atmosphere consisting essentially of an inert gas or a combination of inert gases, wherein the container is airtight. In some embodiments, the airtight container includes a glass vial with a stopper and aluminum cap or a glass ampoule.

Provided herein are kits including a plurality of containers of the above in a cassette and an instruction for medical use. In some embodiments, the kit includes 5 containers.

### BRIEF DESCRIPTION OF THE DRAWINGS

The drawings illustrate certain embodiments of the technology and are not limiting. For clarity and ease of illustration, the drawings are not made to scale and, in some instances, various aspects may be shown exaggerated or enlarged to facilitate an understanding of particular embodiments.

FIG. 1 shows degradation of solid form quercetin composition in air atmosphere under ambient temperature conditions over time;

FIG. 2 shows detection of 2-(3,4-dihydroxybenzoyloxy)-4,6-dihydroxybenzoic acid by chromatography;

FIG. 3 shows identification of 2-(3,4-dihydroxybenzoyloxy)-4,6-dihydroxybenzoic acid by mass spectrometry;

FIG. 4 shows fragmentation of the molecular ion m/z=305 in mass spectrometry using EPI mode;

FIG. 5 shows a schema for the mechanism of formation of 2-(3,4-dihydroxybenzoyloxy)-4,6-dihydroxybenzoic acid from quercetin;

FIG. 6 shows retention times, relative content and UV spectra for 2-(3,4-dihydroxybenzoyloxy)-4,6-dihydroxybenzoic acid and quercetin;

FIG. 7 shows time-dependent accumulation of 2-(3,4-dihydroxybenzoyloxy)-4,6-dihydroxybenzoic acid of solid form quercetin composition in air atmosphere under ambient temperature conditions;

FIG. 8 shows the effect of substitution of air with nitrogen gas storage atmosphere on degradation of a solid form quercetin composition; and,

FIG. 9 shows effect of substitution of air with nitrogen gas storage atmosphere on 2-(3,4-dihydroxybenzoyloxy)-4,6-dihydroxybenzoic acid accumulation of a solid form quercetin composition.

### DETAILED DESCRIPTION OF THE INVENTION

Provided herein are compositions and methods which reduce the degradation of quercetin in solid form quercetin composition and which reduce the formation of contaminants, such as 2-(3,4-dihydroxybenzoyloxy)-4,6-dihydroxybenzoic acid, that forms upon the degradation of solid form quercetin compositions. Also provided are containers that contain solid form quercetin compositions with reduced degradation of quercetin and reduced formation of 2-(3,4-dihydroxybenzoyloxy)-4,6-dihydroxybenzoic acid. Embodiments of the invention can increase the shelf life and patient safety of solid form quercetin compositions.

Applicant surprisingly found that solid form quercetin compositions undergo degradation under ambient storage conditions. Moreover, Applicant also surprisingly found that at least one of the degradation products is a toxic compound, 2-(3,4-dihydroxybenzoyloxy)-4,6-dihydroxybenzoic acid. This byproduct was not predicted by prior studies of quercetin degradation in solution (Wang et al., 2016). Applicant also surprisingly found that by storing solid form quercetin compositions in an inert gas atmosphere, degradation of the product and formation of the toxic byproduct were significantly reduced over a comparable period of time.

Quercetin, 2-(3,4-dihydroxyphenyl)-3,5,7-trihydroxy-4H-chromen-4-one, is a plant flavonoid. Quercetin compositions include relatively pure form quercetin and those that include delivery formulations. Drug delivery refers to approaches, formulations, technologies, and systems for transporting a pharmaceutical compound in the body as needed to safely achieve its desired therapeutic effect. Drug delivery formulations for quercetin can include 1) lipid-based carriers; 2) polymer-based carriers or nanoparticles; 3) inclusion complexes; 4) micelles; and 5) conjugate-based capsulations (Wang et al., 2016). In some embodiments, quercetin compositions include the polymer-based carrier polyvinylpyrrolidone (PVP). In some embodiments, the ratio of PVP:quercetin can be about 8:1, about 9:1, about 10:1, about 11:1, or about 12:1 PVP:quercetin. The relative proportions can vary by about 1%, 2%, 3% or 4%. PVP average molecular weights include, but are not limited to, 8,000, 10,000 or 40,000 g/mol.

In some embodiments, the quercetin composition is in solid form. The solid form of a quercetin composition can be lyophilized (freeze-dried). The solid form of a quercetin composition can be obtained by spray-drying, rotoevaporation or crystallization. When solvent is removed by rotary evaporation, an agglomerated intermediate product is produced, which is then deagglomerated to provide the dry formulation of the quercetin composition. The solid form of a quercetin composition can be provided as a powder, capsules, granules and tablets. In some embodiments, the quercetin composition can be about 9±2% quercetin and about 91±2% polyvinylpyrrolidone w/w in a lyophilizate.

The solution of the quercetin composition can be sterilized with a sterilizing filter prior to preparing the solid form quercetin composition. Typically, this will involve filtering the solution using a solvent compatible 0.2 micron filter to make a sterile solution. The sterile solution can then be aliquoted directly into dose-sized sterile vials or may be aliquoted at a later time, such as in a sterile fill.

A suitable lyophilization cycle can be readily determined by those skilled in the art, as lyophilization conditions may vary. For example, primary drying conditions may vary from −50° C. to −5° C. The length of the cycle is generally known to those skilled in the art, for example, the cycle length may vary from 8 to 48 hours, generally, sufficient time to remove the solvent or liquid from the product. The secondary drying conditions may vary from 0° C. to 50° C.

Quercetin compositions as a lyophilized powder in an air atmosphere were found to undergo time-dependent degradation with a rate of about 2% per year at room temperature (see Example 2). Storage of solid form quercetin compositions under the same conditions in an inert gas atmosphere has a substantially reduced rate of degradation (see Example 4). In some embodiments, the formulation includes a solid form quercetin composition in a vessel in an atmosphere consisting essentially of an inert gas or a combination of inert gases. An inert gas is a gas that is non-reactive. In some embodiments, the inert gas is nitrogen gas. In some embodiments, the inert gas is argon. In other embodiments, the inert gas is a noble gas. Noble gases include, in addition to argon, helium, neon, krypton, xenon and radon. A combination of inert gases is a plurality of inert gases. Non-limiting examples of a combination of inert gases can be 50% nitrogen/50% argon or 95%/nitrogen/5% argon.

In some embodiments, the formulation comprises a solid form quercetin composition in an atmosphere that is essentially oxygen free.

In some embodiments, the solid form quercetin composition comprises greater than 99% of the input quercetin after 24 months at 15-30° C. In some embodiments, the solid form quercetin composition comprises greater than 99% of the input quercetin after 24 months at about 20-25° C. In some embodiments, the solid form quercetin composition comprises greater than 99% of the input quercetin after 24 months at about 21° C. In some embodiments, the solid form quercetin composition comprises more than 97% of the input quercetin after 24 months at 15-30° C. In some embodiments, the solid form quercetin composition comprises more than 97% of the input quercetin after 24 months at 20-25° C. In some embodiments, the solid form quercetin composition comprises more than 97% of the input quercetin after 24 months at about 21° C. In some embodiments, the solid form quercetin composition comprises more than 97.5%, 98%,or 98.5% of the input quercetin after 24 months at 15-30° C. In some embodiments, the solid form quercetin composition comprises more than 97.5%, 98%, or 98.5% of the input quercetin after 24 months at 20-25° C. In some embodiments, the solid form quercetin composition comprises more than 97.5%, 98%, or 98.5% of the input quercetin after 24 months at about 21° C.

Methods for the detection of quercetin, such as by UV spectroscopy, are known in the art. For example, a solid form quercetin composition is dissolved in 96% v/v analytical grade ethanol and measured spectrophotometrically at 374 nm using 96% ethanol as blank control. In parallel, absorbance of reference solutions (with known quercetin concentrations) are measured.

Quercetin degradation, judging by chromatography, leads to formation of several products. One of the degradation products of solid form quercetin compositions is 2-(3,4-dihydroxybenzoyloxy)-4,6-dihydroxybenzoic acid (DB-DBA). This byproduct was not predicted by prior studies of quercetin degradation in solution (Wang et al., 2016) where degradation of solid form quercetin was not systematically studied. Thus, the applicant was the first to identify DB-DBA as a product of solid form quercetin degradation.

Methods to determine toxicity of a compound are known in the art. They include, but are not limited to, in vitro assays for mutagenicity/carcinogenicity (e.g. Ames test in bacteria) and in vitro cytotoxicity (e.g., MTT (e.g. (Salem et al., 2011), MTS assay, SRB, WST-10 or apoptotic assay

(DNA fragmentation, caspase activation (Bhouri et al., 2012a)) in human cells. Acute and chronic toxicity in rodents and non-human primates can also be used (Parasuraman, 2011). Toxicity of water-soluble compounds can be assessed by appropriate model systems of aquatic organisms such as alga, crustacean, fishes and others (Straub, 2002).

Applicant demonstrates herein the toxic effect of DB-DBA. Digallic acid (DGA), a close homolog, is toxic also to a range of human cells. DGA causes apoptosis in human lymphoblastoid cell lines (Bhouri et al., 2012a), and cytotoxicity in other human cell lines (Salem et al., 2011). DGA causes DNA fragmentation in the human lymphoblastoid cell line K562, a hallmark of apoptotic cell death, at concentrations of 200-800 microgram/ml (Bhouri et al., 2012b). DGA causes DNA fragmentation, caspase-3/8 activation and PARP cleavage (other hallmarks of apoptosis) at concentrations of 2.5-10 microgram/ml in human lymphoblastoid TK6 cells. DGA exhibits an IC₅₀ of 8.5 microgram/ml as measured by MTT assay (Bhouri et al., 2012a). The IC₅₀ of DGA by 72-hr MTT assay ranges from 7.3 to 86 microgram/ml in solid human tumor cell lines of different origin (92.1, OCM3, U-87MG, SK-MEL-28, and SK-OV-3). The lowest IC₅₀ of DGA was to melanoma OCM3 cells. DGA is also cytototoxic (with IC₅₀ 47 microgram/ml) to a normal cell line, retinal pigmented epithelial cells (ARPE-19) (Salem et al., 2011).

The chemical formula of DB-DBA differs from DGA by an additional hydroxyl group (underlined) in DGA (see scheme below).

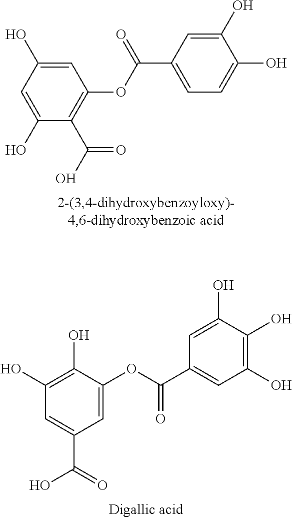 [SMILES: O=C(Oc1cc(O)cc(O)c1C(=O)O)c1ccc(O)c(O)c1 ; O=C(O)c1cc(O)c(O)c(OC(=O)c2cc(O)c(O)c(O)c2)c1]

In some embodiments, the solid form quercetin composition after 24 months at 20-25° C. has a 72 h-EC50 less than about 7.5 milligram/ml in a cytotoxicity assay with MDA-MB231 human breast cancer cells. In some embodiments, the solid form quercetin composition after 24 months at about 21° C. has a 72 h-EC50 less than about 7.5 milligram /ml in cytotoxicity assay with MDA-MB231 human breast cancer cells. In some embodiments, the solid form quercetin composition after 24 months at 20-25° C. has a 72 h-EC50 less than about 750 microgram/ml in a cytotoxicity assay with MDA-MB231 human breast cancer cells. In some embodiments, the solid form quercetin composition after 24 months at about 21° C. has a 72 h-EC50 less than about 750 microgram/ml in cytotoxicity assay with MDA-MB231 human breast cancer cells.

In some embodiments, the solid form quercetin composition comprises less than 1% DB-DBA after 24 months at 20-25° C. In some embodiments, the solid form quercetin composition comprises less than 1% DB-DBA after 24 months at about 21° C. In some embodiments, the solid form quercetin composition comprises less than 0.6%, less than 0.7%, less than 0.8%, or less than 0.9% DB-DBA after 24 months at about 20-25° C. In some embodiments, the solid form quercetin composition comprises less than 0.6%, less than 0.7%, less than 0.8%, less than 0.9% DB-DBA after 24 months at about 21° C. In some embodiments, the solid form quercetin composition comprises less than 0.5% DB-DBA after 24 months at 20-25° C. In some embodiments, the solid form quercetin composition comprises less than 0.5% DB-DBA after 24 months at about 21° C.

Reduction of degradation product of solid form quercetin compositions stored in an inert gas atmosphere results in lowered formation of DB-DBA (see Example 5).

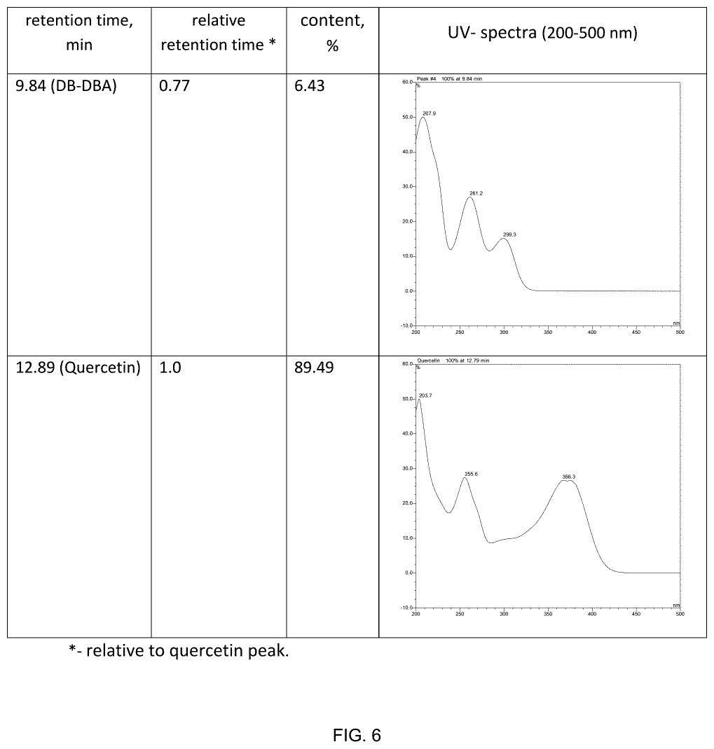

Methods to detect DB-DBA are known in the art, such as by liquid chromatography with UV detection. For example, a solution containing DB-DBA is subjected to liquid chromatography using gradient elution with the following conditions: stainless steel column with stationary phase end-capped octadecylsilyl silica for chromatography R; size (150×3.9) mm, particle size 5 microns. Mobile phase A: 0.1% phosphoric acid v/v; mobile phase B—methanol;column temperature: 25° C.; flow rate: 1 mL/min.; UV detection at 254 nm. Relative retention time is 1.00 for quercetin, and 0.77 for DB-DBA (FIG. 6).

Content of DB-DBA relative to quercetin can be calculated by formula (in percentage):

$${X}_{i} = \frac{{S}_{i}}{{S}_{0}} ,$$

where: Sᵢ—peak area of DB-DBA on the chromatogram of test solution;

S₀—peak area of quercetin on the chromatogram reference solution (a)

Also provided herein are methods of reducing the rate of formation of byproduct by degradation of a solid form quercetin composition by purging air from an airtight container containing the solid form quercetin composition and filling the container with an atmosphere consisting essentially of an inert gas or combination of inert gases. In an embodiment, the method includes displacing the air atmosphere in the container including a solid form quercetin composition with Nitrogen gas. In an embodiment, the method includes displacing the air atmosphere in the container including a solid form quercetin composition with Argon gas. In some embodiments, the byproduct includes DB-DBA.

Also, provided herein are containers that include a solid form quercetin composition and an atmosphere consisting essentially of an inert gas or a combination of inert gases, wherein the container is airtight. Airtight means that gases are not readily exchanged between the inside and outside of the container. Containers include ampoules, vials, syringes, cartridges and bottles. The container can be glass. The glass can be borosilicate glass or soda-lime. The glass can be Type I, II or III. The container can be plastic if airtight. The container can be light-safe (e.g. amber). The container can include a stopper, such as a rubber stopper. The stopper can include a septum for introduction of diluent and for removal of solution from the container. Examples of containers include glass vials with a bromobutyl stopper and an aluminum cap.

### Embodiments

The following are non-limiting embodiments of the invention.

A1. A formulation comprising a solid form quercetin composition in an enclosed atmosphere consisting essentially of an inert gas or a combination of inert gases.

A2. The formulation of embodiment Al, wherein the solid form quercetin composition comprises freeze-dried quercetin.

A3. The formulation of embodiment Al, wherein the solid form quercetin composition is prepared by spray drying, rotoevaporation or crystallization.

A4. The formulation of any one of embodiments A1-A3, wherein the inert gas is essentially oxygen-free.

A5. The formulation of any one of embodiments A1-A4, wherein the inert gas or one of the inert gases in the combination of inert gases is nitrogen.

A6. The formulation of any one of embodiments A1-A4, wherein the inert gas or one of the inert gases in the combination of inert gases is argon.

A7. The formulation of any one of embodiments A1-A6, wherein the solid form quercetin composition further comprises a drug delivery formulation.

A8. The formulation of embodiment A7, wherein the drug delivery formulation is selected from the group consisting of: a lipid-based carrier, a polymer-based carrier, nanoparticles, inclusion complexes, micelles, and a conjugate-based capsulation.

A9. The formulation of embodiment A8, wherein the drug delivery formulation comprises a polymer-based carrier and the polymer-based carrier comprises polyvinylpyrrolidone.

A10. The formulation of embodiment A9, wherein the solid form quercetin composition comprises about 8:1, about 9:1, about 10:1, about 11:1 or about 12:1 polyvinylpyrrolidone:quercetin w/w.

A11. The formulation of embodiment A9, wherein the solid form quercetin composition comprises about 7-11% quercetin and about 89-93% polyvinylpyrrolidone w/w.

A12. The formulation of any one of embodiments A1-A11, wherein the solid form quercetin composition is stored for 24 months at about 20-25° C.

A13. The formulation of embodiment A12, wherein the solid form quercetin composition is stored for 24 months at about 21° C.

A14. The formulation of any one of embodiments A1 to A13, wherein the solid form quercetin composition comprises greater than 97% of the input quercetin.

A15. The formulation of embodiment A14, wherein the solid form quercetin composition comprises greater than 99% of the input quercetin.

A16. The formulation of any one of embodiments A1 to A15, wherein the solid form quercetin composition comprises less than 1% of 2-(3,4-dihydroxybenzoyloxy)-4,6-dihydroxybenzoic acid.

A17. The formulation of embodiment A16, wherein the solid form quercetin composition comprises less than 0.9, 0.8, 0.7, 0.6, or 0.5% of 2-(3,4-dihydroxybenzoyloxy)-4,6-dihydroxybenzoic acid.

A18. The formulation of any one of embodiments Al to A17, wherein the solid form quercetin composition has 72 h-EC50 less than 7.5 mg/ml in a cytotoxicity assay using MDA-MB231 human breast cancer cells.

A19. The formulation of embodiment A18, wherein the solid form quercetin composition has a 72 h-EC50 less than 750 microgram/ml.

B1. A formulation comprising a solid form quercetin composition, wherein the solid form quercetin composition comprises less than 1% 2-(3,4-dihydroxybenzoyloxy)-4,6-dihydroxybenzoic acid after 24 months storage at about 20-25° C.

B2. The formulation of embodiment B1, wherein the solid form quercetin composition comprises less than 1% 2-(3,4-dihydroxybenzoyloxy)-4,6-dihydroxybenzoic acid after 24 months storage at about 21° C.

B3. The formulation of embodiment B1 or B2, wherein the solid form quercetin composition comprises less than 0.9%, 0.8%, 0.7%, 0.6%, or 0.5% 2-(3,4-dihydroxybenzoyloxy)-4,6-dihydroxybenzoic acid.

B4. The formulation of any one of embodiments B1-B3, wherein the solid form quercetin composition comprises freeze-dried quercetin.

B5. The formulation of any one of embodiments B1-B3, wherein the solid form quercetin composition is prepared by spraying drying, rotoevaporation, or crystallization.

B6. The formulation of any one of embodiments B1-135, further comprising an enclosed atmosphere consisting essentially of an inert gas or a combination of inert gases.

B7. The formulation of embodiment B6, wherein the inert gas or combination of inert gases is essentially oxygen-free.

B8. The formulation of embodiment B6, wherein the inert gas or one of the inert gases of the combination of inert gases is nitrogen.

B9. The formulation of embodiment B6, wherein the inert gas or one of the inert gases of the combination of inert gases is argon.

B10. The formulation of any one of embodiments B1-B9, wherein the solid form quercetin composition further comprises a drug delivery formulation.

B11. The formulation of embodiment B10, wherein the drug delivery formulation is selected from the group consisting of: a lipid-based carrier, a polymer-based carrier, nanoparticles, inclusion complexes, micelles, and a conjugate-based capsulation.

B12. The formulation of embodiment B11, wherein the drug delivery formulation comprises a polymer-based carrier and the polymer-based carrier comprises polyvinylpyrrolidone.

B13. The formulation of embodiment B12, wherein the solid form quercetin composition comprises about 8:1, about 9:1, about 10:1, about 11:1, or about 12:1 polyvinylpyrrolidone:quercetin w/w.

B14. The formulation of embodiment B12, wherein the solid form quercetin composition comprises about 7-11% quercetin and 89-93% polyvinylpyrrolidone w/w.

B15. The formulation of any one of embodiments B1-B14, wherein the solid form quercetin composition comprises greater than 97% of the input quercetin.

B16. The formulation of embodiment B15, wherein the solid form quercetin composition comprises greater than 99% of the input quercetin.

B17. The formulation of any one of embodiments B1-B16, wherein the solid form quercetin composition has a 72 h-EC50 less than 7.5 mg/ml in a cytotoxicity assay using MDA-MB231 human breast cancer cells.

B18. The formulation of embodiment B17, wherein the solid form quercetin composition has a 72 h-EC50 less than 750 microgram/ml.

C1. A container comprising a formulation according to any one of embodiments A1-A19 or B1-B18 in a vessel, wherein the container is airtight.

C2. The container of embodiment C1, wherein the airtight container comprises a glass vial with a stopper and aluminum cap.

C3. The container of embodiment C1, wherein the airtight container comprises a glass ampoule

D1. A kit comprising a plurality of containers according to any one of embodiments C1 to C3 in a cassette.

D2. The kit of embodiment D1 comprising 5 containers.

E1. A method of reducing the rate of formation of a toxic contaminant by degradation of a solid form quercetin composition comprising purging air from an airtight container containing the solid form quercetin composition and filling the container with an atmosphere consisting essentially of an inert gas or a combination of inert gases.

E2. The method of embodiment E1, wherein the solid form quercetin composition comprises freeze-dried quercetin.

E3. The method of embodiment E1, wherein the solid form quercetin composition is prepared by spray drying, rotoevaporation or crystallization.

E4. The method of any one of embodiments E1-E3, wherein the inert gas is essentially oxygen-free.

E5. The method of any one of embodiments E1-E4, wherein the inert gas or one of the inert gases in the combination of inert gases is nitrogen.

E6. The method of any one of embodiments E1-E4, wherein the inert gas or one of the inert gases in the combination of inert gases is argon.

E7. The method of any one of embodiments E1-E6, wherein the solid form quercetin composition further comprises a drug delivery formulation.

E8. The method of embodiment E7, wherein the drug delivery formulation is selected from the group consisting of: a lipid-based carrier, a polymer-based carrier, nanoparticles, inclusion complexes, micelles, and a conjugate-based capsulation.

E9. The method of embodiment E8, wherein the drug delivery formulation comprises a polymer-based carrier and the polymer-based carrier comprises polyvinylpyrrolidone.

E10. The method of embodiment E9, wherein the solid form quercetin composition comprises about 8:1, about 9:1, about 10:1, about 11:1 or about 12:1 polyvinylpyrrolidone:quercetin w/w.

E11. The method of embodiment E9, wherein the solid form quercetin composition comprises 7-11% quercetin and 89-93% polyvinylpyrrolidone w/w.

E12. The method of any one of embodiments E1-E11, wherein the contaminant comprises 2-(3,4-dihydroxybenzoyloxy)-4,6-dihydroxybenzoic acid.

E13. The method of any one of embodiments E1-E12, wherein the airtight container comprises a glass vial with a stopper and aluminum cap.

E14. The method of any one of embodiments E1-E12, wherein the airtight container comprises a glass ampoule.

E15. The method of any one of embodiments E1-E14, comprising determining the level of the toxic contaminant.

E16. The method of embodiment E15, wherein the toxic contaminant comprises 2-(3,4-dihydroxybenzoyloxy)-4,6-dihydroxybenzoic acid.

F1. A method of reducing the degradation of a solid form quercetin composition comprising purging air from an airtight container containing the solid form quercetin composition and filling the container with an atmosphere consisting essentially of an inert gas or a combination of inert gases.

F2. The method of embodiment F1, wherein the solid form quercetin composition comprises freeze-dried quercetin.

F3. The method of embodiment F1, wherein the solid form quercetin composition is prepared by spraying drying, rotoevaporation or crystallization.

F4. The method of any one of embodiments F1-F3, wherein the inert gas is essentially oxygen-free.

F5. The method of any one of embodiments F1-F4, wherein the inert gas or one of the inert gases of the combination of inert gases is nitrogen.

F6. The method of any one of embodiments F1-F4, wherein the inert gas or one of the inert gases of the combination of inert gases is argon.

F7. The method of any one of embodiments F1-F6, wherein the solid form quercetin composition further comprises a drug delivery formulation.

F8. The method of embodiment F7, wherein the drug delivery formulation is selected from the group consisting of: a lipid-based carrier, a polymer-based carrier, nanoparticles, inclusion complexes, micelles, and a conjugate-based capsulation.

F9. The method of embodiment F8, wherein the drug delivery formulation comprises a polymer-based carrier and the polymer-based carrier comprises polyvinylpyrrolidone.

F10. The method of embodiment F9, wherein the solid form quercetin composition comprises about 8:1, about 9:1, about 10:1, about 11:1, or about 12:1 polyvinylpyrrolidone:quercetin w/w.

F11. The method of embodiment F9, wherein the solid form quercetin composition comprises 7-11% quercetin and 89-93% polyvinylpyrrolidone w/w.

F12. The method of any one of embodiments F1-F11, wherein the solid form quercetin composition comprises greater than 97% of the input quercetin after 24 months at about 20-25° C.

F13. The method of embodiment F12, wherein the solid form quercetin composition comprises greater than 97% of the input quercetin after 24 months at about 21° C.

F14. The method of any one of embodiments F12 or F13, wherein the solid form quercetin composition comprises greater than 99% of the input quercetin.

F15. The method of any one of embodiments F1-F14, wherein the airtight container comprises a glass vial with a stopper and aluminum cap.

F16. The method of any one of embodiments F1-F14, wherein the airtight container comprises a glass ampoule.

### REFERENCES

Bhouri, W., J. Boubaker, I. Skandrani, K. Ghedira, and L. Chekir Ghedira. 2012a. Investigation of the apoptotic way induced by digallic acid in human lymphoblastoid TK6 cells. Cancer Cell International. 12:26.

Bhouri, W., I. Skandrani, M.b. Sghair, M.-G. D. Franca, K. Ghedira, and L. C. Ghedira. 2012b. Digallic acid from Pistascia lentiscus fruits induces apoptosis and enhances antioxidant activities. Phytotherapy Research. 26:387-391.

Dachineni, R., D. R. Kumar, E. Callegari, S. S. Kesharwani, R. Sankaranarayanan, T. Seefeldt, H. Tummala, and G. J. Bhat. 2017. Salicylic acid metabolites and derivatives inhibit CDK activity: Novel insights into aspirin's chemopreventive effects against colorectal cancer. International Journal of Oncology 51:1661-1673.

Denina, R. 2013. Heart Failure Treatment In Patients With Recurrent Myocardial Infarction. The Pharma Innovation 2:30-35.

Graefe, E., W. Joerg, M. Silke, R. Anne-Kathrin, U. Bernhard, D. Bernd, P. Holger, J. Gisela, D. Hartmut, and V. Markus. 2001. Pharmacokinetics and Bioavailability of Quercetin Glycosides in Humans. The Journal of Clinical Pharmacology 41:492-499.

Harwood, M., B. Danielewska-Nikiel, J. F. Borzelleca, G. W. Flamm, G. M. Williams, and T. C. Lines. 2007. A critical review of the data related to the safety of quercetin and lack of evidence of in vivo toxicity, including lack of genotoxic/carcinogenic properties. Food and Chemical Toxicology 45:2179-2205.

Kamaya, Y., Y. Fukaya, and K. Suzuki. 2005. Acute toxicity of benzoic acids to the crustacean Daphnia magna. Chemosphere 59:255-261.

Kuzmenko, M. A., V. B. Pavlyuchenko, L. V. Tumanovskaya, V. E. Dosenko, and A. A. Moybenko. 2013. [Experimental therapy of cardiac remodeling with quercetin-containing drugs]. Patol Fiziol Eksp Ter: 17-22.

Lee, P. Y., and C. Y. Chen. 2009. Toxicity and quantitative structure—activity relationships of benzoic acids to Pseudokirchneriella subcapitata. Journal of Hazardous Materials 165:156-161.

Martins, J., L. Oliva Teles, and V. Vasconcelos. 2007. Assays with Daphnia magna and Danio rerio as alert systems in aquatic toxicology. Environment International. 33:414-425.

Parasuraman, S. 2011. Toxicological screening. Journal of Pharmacology & Pharmacotherapeutics 2:74-79.

Pashevin, D. A., L. V. Tumanovska, V. E. Dosenko, V. S. Nagibin, V. L. Gurianova, and A. A. Moibenko. 2011. Antiatherogenic effect of quercetin is mediated by proteasome inhibition in the aorta and circulating leukocytes. Pharmacol Rep. 63:1009-18.

Porcu, E. P., M. Cossu, G. Rassu, P. Giunchedi, G. Cerri, J. Pourová, I. Najmanová, T. Migkos, V. Pilařová, L. Nováková, P. Mladěnka, and E. Gavini. 2018. Aqueous injection of quercetin: An approach for confirmation of its direct in vivo cardiovascular effects. International Journal of Pharmaceutics 541:224-233.

Ryzhkova, N. O., T. I. Gavrilenko, and O. M. Parkhomenko. 2016. Korvitin Reduces the High Maintenance of Myeloperoxidase in Plasma of Blood of Patients with the Acute Infarct of Myocardium. Fiziol Zh. 62:87-93.

Salem, M. M., F. H. Davidorf, and M. H. Abdel-Rahman. 2011. In vitro anti-uveal melanoma activity of phenolic compounds from the Egyptian medicinal plant Acacia nilotica. Fitoterapia. 82:1279-1284.

Straub, J. O. 2002. Environmental risk assessment for new human pharmaceuticals in the European Union according to the draft guideline/discussion paper of January 2001. Toxicology Letters 131:137-143.

Wang, C.-C., L.-G. Chen, and L.-L. Yang. 2002. Cytotoxic Activity of Sesquiterpenoids from Atractylodes ovata on Leukemia Cell Lines. Planta Med. 68:204-208.

Wang, W., C. Sun, L. Mao, P. Ma, F. Liu, J. Yang, and Y. Gao. 2016. The biological activities, chemical stability, metabolism and delivery systems of quercetin: A review. Trends in Food Science & Technology 56:21-38.

### EXAMPLES

### Example 1

### Preparation of Lyophilized 90%/10% PVP/Guercetin in Nitrogen Atmosphere

### Preparation of Intermediate Product Solution

Preparation of alcohol solution (dissolution of quercetin and polyvinylpyrrolidone (PVP) in ethanol) and its evaporation (the formation of a homogeneous dry basis) were carried out with a rotary evaporator (Strike 5000, Steroglass, Perugia, Italy). 25 L Ethanol 96% (high-purity solvent, SE “Ukrspirt”, Lipniki, Ukraine), 1.0 kg quercetin (high-purity solvent, SE “Ukrspirt”, Lipniki, Ukraine) and 9.01 kg polyvinylpyrrolidone (PVP) (EP grade, BASF SE, Ludwigshafen, Germany) were loaded into a 100 liter round-bottomed flask of the rotary evaporator. Dissolution was performed using the following parameters:

| | | |
|---|---|---|
| Temperature | (70 ± 5) | ° C. |
| of water-bath | | |
| Vacuum level | 800 | mbar |
| Rate of stirring | 50-100 | rpm |
| Duration | 3.0 | hours |

Stirring continued until the components were completely dissolved (visual control).

Evaporation of alcohol solution (obtaining of dry basis). Upon dissolution, the vacuum level was gradually increased at such a rate to maintain boiling of the solution. According to evaporation of solution the speed of rotation of flask was reduced. The evaporation continued until dryness.

| | | |
|---|---|---|
| Temperature of water-bath | (70 ± 5) | ° C. |
| Vacuum level at the | 800 | mbar |
| beginning of evaporation | | |
| Vacuum level at the | 24-26 | mbar |
| end of evaporation | | |
| Rate of stirring | 50-100 | rpm |
| Duration of evaporation | (7.0-7.5) | hours |

A sodium hydroxide solution was prepared by charging a reactor (PCBF100, OLSA, Milan, Italy) with 13.5 L water for injection and 41.5 g sodium hydroxide (pharma grade EP, SPOLCHEMIE, Czech Republic) and stirred until complete dissolution. The rotational speed of the mixer was 295-300 rpm and the dissolution time was approximately 5 minutes.

Preparation of Aqueous Solution

The dry basis, (obtained at the stage of evaporation of alcohol solution), was dissolved in 52.0 L water for injection. After dissolution of the mass, a reactor (TK001 PCBF50, OLSA, Milan, Italy) was charged with the sodium hydroxide solution using a peristaltic pump (MASTER-FLEX LS 77301-20, MASTER-FLEX, Vernon Hills, USA) to adjust the pH of the solution to about 6.7-7.2. The resulting intermediate product solution was prefiltered using a cartridge filter with a pore size of 0.20 micron (DA36MDMM002MCY2, DANMIL A/S, Greve, Denmark).

Filling of the Vials

The intermediate product solution was tested for microbial load on a filter pursuant to standard methods.

Glass vials (cat#0111075.1063, Medical Glass, Bratislava, Slovak Republik) were filled with a solution of the intermediate product on a filling and capping machine using a sterile filter-capsule with a heterogenous cellulose acetate double layer having filters of 0.45 and 0.22 micron pore size (SARTOBRAN Pe, Sartorius Stedim Biotech GmbH, Gottingen, Germany).

The volume of filled intermediate product solution was approximately 3.6-4.2 ml. The filled vials were topped with rubber stoppers (cat#C5919, Aptar Stelmi SAS, Granville, France) in vented position and transferred to a transport laminar trolley (LF 0.6×0.9, CHRIST, Osterode am Harz, Germany) and passed to the lyophilization process.

Lyophilyzation (Sublimation) of the Intermediate Product Solution

Drying of the intermediate product solution was performed in a lyophilizer (EPSILON 2-45 DS, CHRIST, Osterode am Harz, Germany) according to manufacturer instruction. After lyophilization, the vials were removed from the lyophilizer, nitrogen gas was introduced into each vial using a Nitrogen generator (MAXIGAS 108ECALL, PARKER HANNIFIN SP ZOO, Warsaw, Poland), and then the rubber stoppers tightly closed.

Sealing (Packing) of Vials

The stoppered vials were capped with aluminum caps (cat# K-2-20, Chernivets'kyy Zavod Medychnykh Vyrobiv, Chernivtsi, Ukraine) on a filling and capping machine. Sealing (packing), the hermeticity, and the quality of the lyophilized product were tested in accordance with QC procedures.

Packaging and Labeling

Vial labeling was performed on a labeling machine.

Labeled vials were placed manually into a cassette, 5 vials per cassette. Each cassette together with instruction for medical use was put into a case.

Cases were placed into boxes together with the label “Packer”. The box was covered with adhesive tape (with a logo). A group label printed with the batch number and expiration date was glued with a transparent tape

### Example 2

A Solid Form Quercetin Composition in an Air Atmosphere Undergoes Degradation in Ambient Temperature Storage

Vials of CORVITIN®, a medical formulation of quercetin (10%)/PVP (90%) wherein the solid form quercetin composition is in an air atmosphere, were stored at room temperature (21° C.) and samples were taken at indicated time points (0, 6, 12, 18, 24 and 36 months).

Test solution. 400.0 mg of vial contents were dissolved in 100 ml 96% (v/v) ethanol. 2.0 ml of the solution was diluted to 100.0 ml with 96% (v/v) ethanol.

Reference solution. 40.0 mg of working standard of quercetin (assay: 97.5%-101.5%, PJSC SIC “Borshchahivskiy CPP”, Kiev, Ukraine) was dissolved in 100 ml 96% (v/v) ethanol. 2.0 ml of the solution was diluted to 100.0 ml with 96% (v/v) ethanol.

Absorbances of the test solution and of the reference solution were measured spectrophotometrically at 374 nm using a cell with a 10-mm path length wherein 96% (v/v) ethanol was used as a blank.
Vial content of quercetin (X₁) was determined by the formula:

$${X}_{1} = \frac{{A}_{1} * {m}_{0} * 100 * 100 * 2 * b * P}{{A}_{0} * {m}_{1} * 100 * 2 * 100 * 100} = \frac{{A}_{1} * {m}_{0} * b * P}{{A}_{0} * {m}_{1} * 100} ,$$

where A₁—the absorbance of the test solution;
A₀—the absorbance of the reference solution;
m₁—the weight of the sample of the preparation, milligrams;
m₀—the weight of the sample of quercetin reference solution, milligrams;
b—the average vial contents, milligrams;
P—the content of quercetin in quercetin RS, per cent.

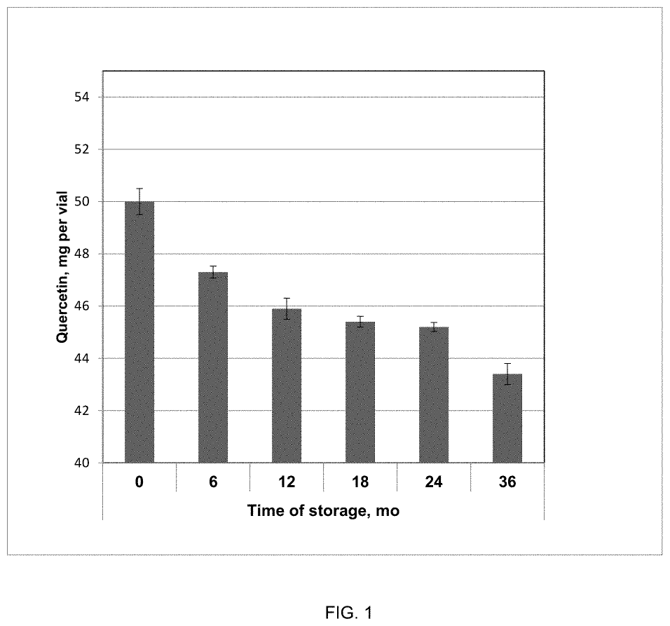

Data from 3 independent experiments are shown in FIG. 1. Based on these experiments, quercetin content in CORVITIN® decreases gradually during storage at a rate of about 2.3% per 12 months.

### Example 3

Formation of 2-(3,4-dihydroxybenzoyloxy)-4,6-dihydroxybenzoic Acid (DB-DBA) as a Result of Quercetin Degradation

Vials of CORVITIN® (50 milligram/vial) were stored for 6 months at 40° C.

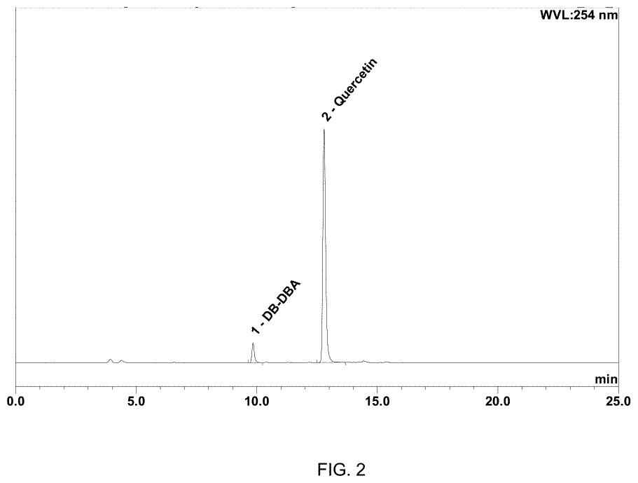

Samples of CORVITIN® were analyzed by liquid chromatography using a Dionex UltiMate 3000 HPLC system (Thermo Fisher Scientific, Inc., Waltham, Mass.) with DAD and MS detectors (3200 QTRAP System (AB Sciex LLC, Framingham, Mass.)) connected consecutively. DAD, MS-detector. Ionization ESI and operating mode Q3 and EPI were used. Flavonols, such as quercetin, are easily deprotonated allowing for facile ionization and strong signals at trace amounts in the negative mode. Q3—full scan mode allows recording of the MS spectra in a given range (in this case, 50-1000 m/z) at each point of the chromatogram. It also allows establishing the m/z ratio for the molecular ion. EPI (Enhance Product Ion Scan) was used to obtain the mass of fragments formed during fragmentation of the molecular ion with a given m/z ratio. Linear Ion Trap (LIT) scan mode was used for accumulating fragments to obtain a MS spectra with high-intensity and high resolution. FIG. 2 shows a chromatogram of compounds formed during degradation of quercetin. One of them, marked DB-DBA, was identified as 2-(3,4-dihydroxybenzoyloxy)-4,6-dihydroxybenzoic acid (DB-DBA) by liquid chromatography-mass spectroscopy (LC-MS) as described below.

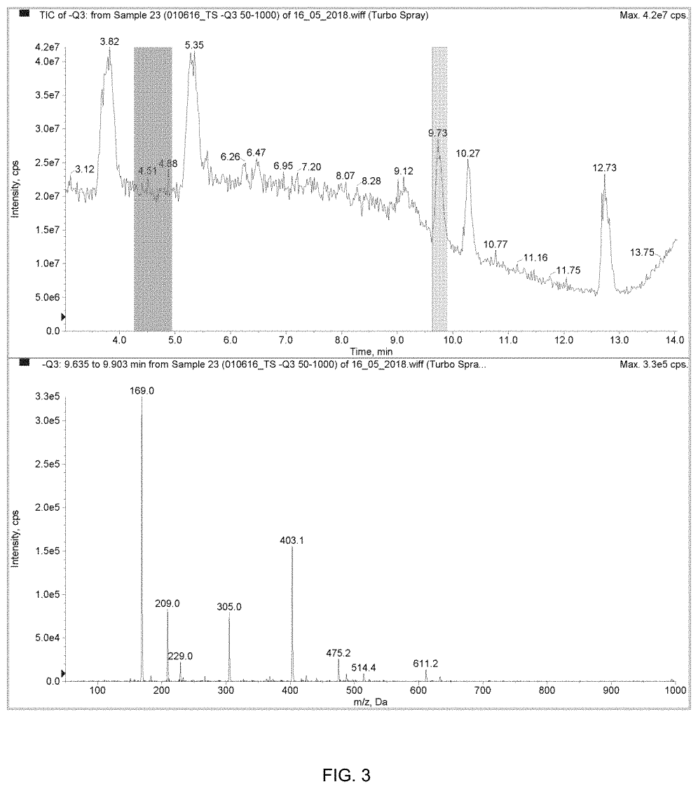

The MS spectrum of the chromatograph peak with a retention time 9.73 min, (relative retention 0.77) is shown in FIG. 3. When electrospray ionization with a negative polarity is used, the molecular ion [M−H]−m/z=305 is formed and a peak [2M−H]−m/z=611 (dimer anion) is also observed. Also observed in the mass spectrum is a cluster of peaks of an unknown compound formed under ionization conditions with m/z=(305+98)=403.

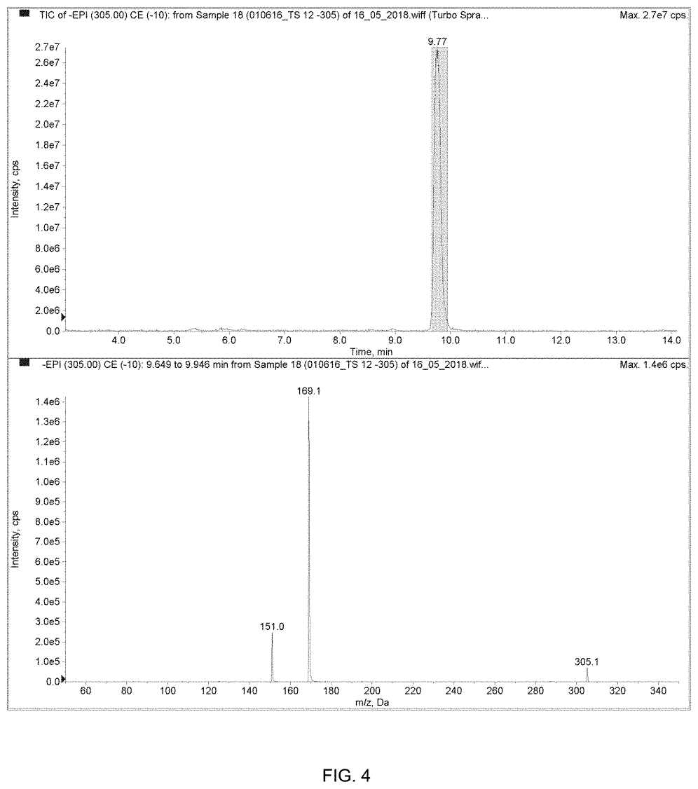

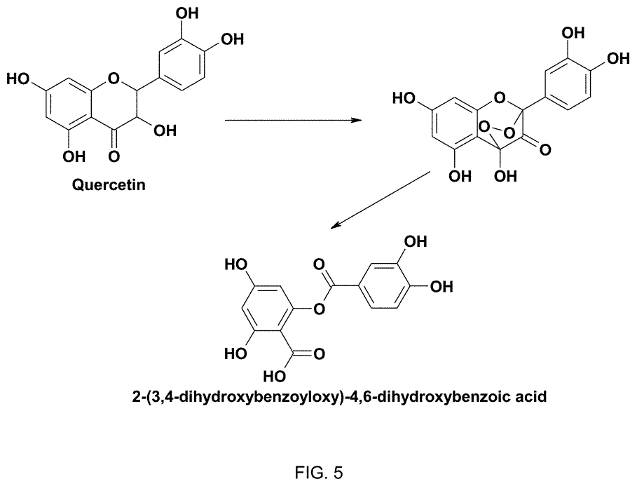

The fragmentation of the molecular ion m/z=305 was determined using EPI mode, negative polarity, with a collision energy −10 V (FIG. 4). The fragmentation shows that the peak with a relative retention 0.77 corresponds to 2-[3,4-dihydrobenzol)oxy]-4,6-dihydrobenzoic acid (DB-DBA). This compound is a product of quercetin degradation through the formation of intermediate compound cyclic peroxide quercetin. FIG. 5 shows the proposed chemical mechanism of formation of 2-(3,4-dihydroxybenzoyloxy)-4,6-dihydroxybenzoic acid during quercetin degradation.

FIG. 6 shows retention times, UV spectra and structures of DB-DBA and quercetin. DB-DBA has a relative retention time (RRT) 0.77, whereas the RRT for quercetin is 1.0. Major absorption peaks of DB-DBA are 207.9, 261.2, and 299.3 nm while those of quercetin are 203.7, 255.6 and 366 nm.

Vials of 10% quercetin/90% PVP were kept at room temperature (21° C.). Samples were removed at indicated time points and assayed for content of DB-DBA by chromatography as described below. For test solution contents of one vial with 70 ml of 96% ethanol was transferred to a 100 ml volumetric flask, diluted to 100 ml with the same solvent and mixed. For reference solution (a), 1.0 ml of test solution was placed in a 100 ml volumetric flask, diluted to 100 ml with 96% ethanol and mixed. For reference solution (b) 10.0 mg working standard of quercetin for system suitability (containing isorhamnetin and kaempferol, PJSC SIC “Borshchahivskiy CPP”, Kiev, Ukraine) was dissolved in 30 ml of 96% ethanol, diluted to 50 ml with the same solvent and mixed. Chromatography was performed on a Dionex HPLC with UV detector using gradient elution with the following conditions:
Stainless steel column with stationary phase end-capped octadecylsilyl silica for chromatography; size (150×3.9) mm, particle size 5 microns
Mobile phase A: 1.0 mL of phosphoric acid diluted to 1000 mL with water for chromatography, mixed and degassed;
Mobile phase B: methanol gradient grade;
Column temperature: 25° C.
Flow rate: 1 mL/min;
Detection at 254 nm;
Injection volume:10 microL.

Gradient Program:

| | Mobile | Mobile |
|---|---|---|
| Time, | phase A, | phase B, |
| min | % (V/V) | % (V/V) |
| 0 | 80 | 20 |
| 1 | 80 | 20 |
| 16 | 20 | 80 |
| 18 | 20 | 80 |
| 19 | 80 | 20 |
| 25 | 80 | 20 |

Injected reference solution (b).

Chromatography system was considered to be suitable if the following requirements are performed:

resolution: minimum 2.0 between the principal peak due to quercetin and the peak due to kaempferol.
Inject test solution and reference solution (a).
Peaks of kaempferol and DB-DBA were determined by relative retention times:

| Name of | Relative |
|---|---|
| product | retention |
| DB-DBA | 0.77 |
| quercetin | 1.00 |
| kaempferol | 1.11 |

Content of DB-DBA was calculated by formula (in percentages):

$${X}_{i} = \frac{{S}_{i}}{{S}_{0}} ,$$

where: Sᵢ—peak area of DB-DBA on the chromatogram of test solution;
S₀—peak area of quercetin on the chromatogram of reference solution (a);
Reporting threshold: 0.05%.

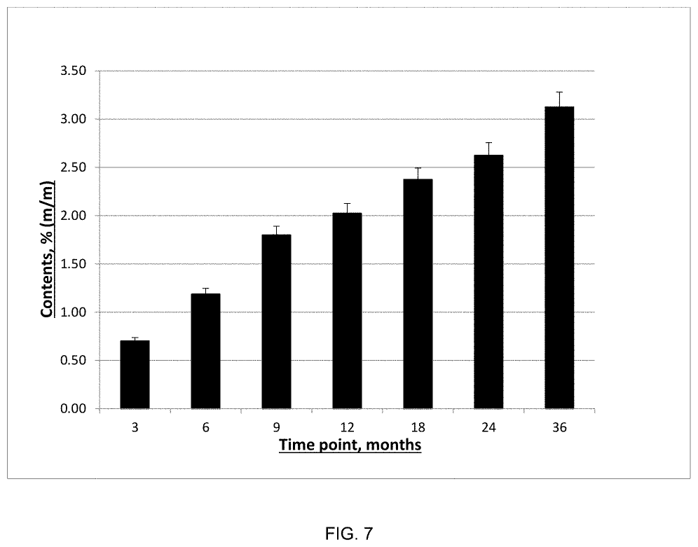

Data from 3 independent experiments are shown. During storage at room temperature, DB-DBA was gradually accumulated at an approximate rate of 1.25% of quercetin (w/w) per 12 months (FIG. 7).

### Example 4

Substitution of Air with Nitrogen Gas in Container with Solid form Quercetin Composition Reduces Quercetin Degradation at Ambient Temperature

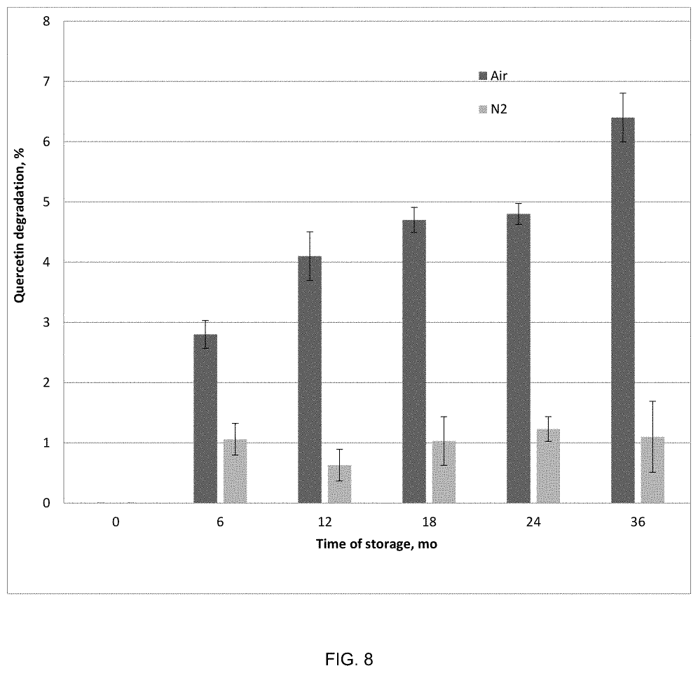

Vials containing (group 1) a lyophilized quercetin (10%)/PVP (90%) composition in an air atmosphere and (group 2) vials containing a lyophilized quercetin (10%)/PVP (90%) composition in a nitrogen gas atmosphere were stored at room temperature. Samples were obtained for each group at the indicated time points (0, 6, 12, 18, 24 and 36 months) and quercetin was assayed by spectrophotometrically at 374 nm and the amount of quercetin remaining calculated as average mass per vial as described above and the percent degradation was calculated for each. Vials containing (group 1) a lyophilized quercetin (10%)/PVP (90%) composition in an air atmosphere showed a significantly greater rate of degradation of quercetin than (group 2) vials containing a lyophilized quercetin (10%)/PVP (90%) composition in a nitrogen gas atmosphere (FIG. 8). Degradation of quercetin was reduced by approximately 80% when the lyophilized quercetin (10%)/PVP (90%) composition was stored in a nitrogen gas atmosphere compared to an air atmosphere at the same ambient storage temperature.

### Example 5

Substitution of Air with Nitrogen Gas in Container with Solid Form Quercetin Composition Reduces Formation of DB-DBA at Ambient Temperature

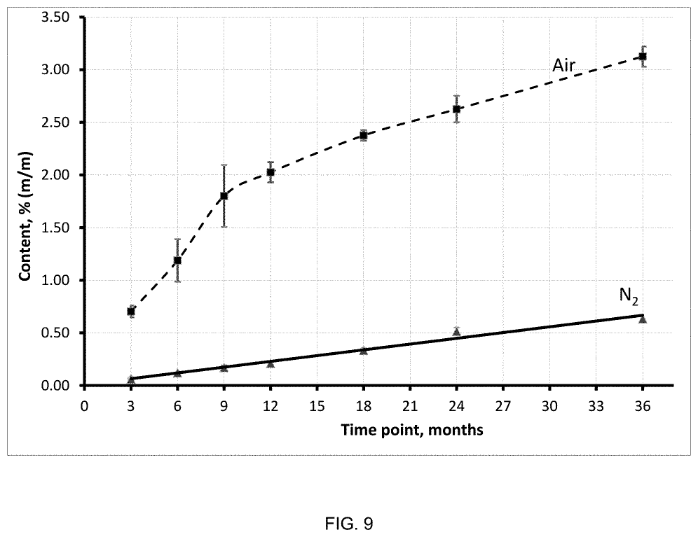

Vials containing (group 1) a lyophilized quercetin (10%)/PVP (90%) composition in an air atmosphere and (group 2) vials containing a lyophilized quercetin (10%)/PVP (90%) composition in a nitrogen gas atmosphere were stored at room temperature. Samples were obtained for each group at the indicated time points (0, 3, 6, 9, 12, 18, 24 and 36 months) and assayed for content of DB-DBA by chromatography as described above. Data from 3 independent experiments are shown. Substitution of air with nitrogen reduced DB-DBA accumulation by approximately 80% (FIG. 9).

### Example 6

Determination of Toxicity of 2-(3,4-dihydroxybenzoyloxy)-4,6-dihydroxybenzoic Acid

Toxicity of DB-DBA (2-(3,4-dihydroxybenzoyloxy)-4,6-dihydroxybenzoic acid) was assessed in 96-well plates (Thermofisher, Walthman, Mass.) on 1×10³ MDA-MB231 human breast cancer cells/well. DB-DBA was diluted to concentrations from 500 to 20 ug/ml. The plate was incubated at 37° C. for 72 h. Cell viability was measured in a CellTiter 96® AQueous One Solution Cell Proliferation Assay (Promega, Madison, Wis.), according to the manufacturer's protocol. The absorbance at 490 nm is determined spectrophotometrically and IC50 was calculated to be 75 ug/ml.

| DB-DBA, | | |
|---|---|---|
| microgram/ | Viability, | SD, |
| ml | % | % |
| 0 | 100 | 0 |
| 20 | 95 | 5 |
| 50 | 62 | 7 |
| 100 | 41 | 5 |
| 250 | 19 | 3 |
| 500 | 10 | 3 |

### Example 7

Determination of Toxicity of Solid Form Quercetin Composition Using Cell Viability Assay in Human Breast Cancer Cells

A sample of CORVITIN (lyophilized 10% quercetin/90% polyvinylpyrrolidone) is resuspended in medium and introduced at increasing dilutions (from 10 to 0.5 microliters per 100 microliter) to a 96-well plate (Thermofisher, Walthman, Mass.) on 1×10³ MDA-MB231 human breast cancer cells/well. The plate is incubated at 37° C. for 72 h. Cell viability is assessed in a CellTiter 96® AQueous One Solution Cell Proliferation Assay (Promega, Madison, Wis.), according to the manufacturer's protocol. The absorbance at 490 nm is determined spectrophotometrically. The IC50 is calculated based on the reduction of viability to increasing quercetin concentration.

### Example 8

Determination of Toxicity of Solid Form Quercetin Composition Using DNA Fragmentation Assay in K562 Human Cells

DNA fragmentation is analyzed by agarose gel electrophoresis as described (Wang et al., 2002) with slight modifications (Bhouri et al., 2012b). K562 cells (1.5 10⁶ cells/mL) are exposed to samples of quercetin (10%) with PVP (90%) which is resuspended and introduced at increasing dilutions to the wells for 24 and 48 h, and then harvested by centrifugation. Control cells are treated with 0.5% of DMSO. Cell pellets are resuspended in 200 microL of lysis buffer (50 mM Tris-HCl, pH8.0, 10 mM EDTA, 0.5% N-lauroyl sarcosine sodium salt) at room temperature for 1 h, then centrifuged at 12,000 g for 20 min at 4° C. The supernatant is incubated overnight at 56° C. with 250 μg/mL proteinase K. Cell lysates are then treated with 2 mg/ml RNase A and incubated at 56° C. for an additional 2 h. DNA is extracted with chloroform/phenol/isoamylalcohol (24:25:1, v/v/v), precipitated by addition of ethanol, and separated from the aqueous phase by centrifugation at 14,000 g for 30 min at 4° C. The recovered aqueous solution is transferred to a 1.5% agarose gel and electrophoresis is carried out at 67V for ¾ h with TAE (Tris 2M, sodium acetate 1M, EDTA 50 mM) as the running buffer. DNA in the gel is visualized under UV light (Bhouri et al., 2012b).

### Example 9

Determination of Toxicity of Solid Form Quercetin Composition Using Cytotoxicity in TK6 Human Cell Line

Cytotoxicity is determined by MTT [3-(4,5-dimethylthiazol-2-yl)-2,5-diphenyltetrazolium bromide] assay (Cat. No. 11 465 007 001, Sigma, Mo., USA) as described (Bhouri et al., 2012a). Human lymphoblastoid TK6 cells are seeded in 96-well plates overnight at a density of 5×10⁴ cells/well according to manufacturer's instructions. A sample of quercetin (10%) with PVP (90%) is resuspended and introduced at increasing dilutions to the wells. The plates are incubated for 24 h and optical density of formazan product formed is determined as described (Bhouri et al., 2012a).

### Example 10

Determination of Toxicity of Solid Form Quercetin Composition Using Cytotoxicity in OCM3 Human Cell Line.

A cell cytotoxicity assay is performed in 96-well plate on 1 to 3×10³ cells/well as described (Salem et al., 2011). A sample of solid form quercetin (10%)/PVP (90%) composition is resuspended and introduced at increasing dilutions to the wells for 72 h. Cell viability is assessed in a CellTiter 96® AQueous One Solution Cell Proliferation Assay (Promega, Madison, Wis.), according to the manufacturer's protocol. The absorbance at 490 nm is determined spectrophotometrically.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

The above disclosure is intended only to convey an understanding of the present invention to those skilled in the art, and is not intended to be limiting. It will be appreciated that various modifications to the disclosed embodiments are possible without departing from the scope of the invention. Therefore, the scope of the present invention should be construed solely by reference to the appended claims.

1-42. (canceled)

43. A method of reducing the rate of formation of a toxic contaminant by degradation of a freeze-dried quercetin composition comprising purging air from an airtight container containing the freeze-dried quercetin composition and filling the container with an atmosphere consisting essentially of an inert gas or a combination of inert gases, wherein the freeze-dried quercetin composition further comprises a drug delivery formulation.

44-46. (canceled)

47. The method of claim 43, wherein the inert gas or one of the inert gases in the combination of inert gases is nitrogen.

48-49. (canceled)

50. The method of claim 43, wherein the drug delivery formulation is selected from the group consisting of: a lipid-based carrier, a polymer-based carrier, nanoparticles, inclusion complexes, micelles, and a conjugate-based capsulation.

51. The method of claim 50, wherein the drug delivery formulation comprises a polymer-based carrier and the polymer-based carrier comprises polyvinylpyrrolidone.

52. (canceled)

53. The method of claim 51, wherein the solid form quercetin composition comprises 9±2% quercetin and about 91±2% polyvinylpyrrolidone w/w.

54. The method of claim 43, wherein the contaminant comprises 2-(3,4-dihydroxybenzoyloxy)-4,6-dihydroxyb enzoic acid.

55. The method of claim 43, wherein the airtight container comprises a glass vial with a stopper and aluminum cap.

56-72. (canceled)

73. The method of claim 43, wherein the inert gas is essentially oxygen-free.

74. The method of claim 43, wherein the inert gas or one of the inert gases in the combination of inert gases is argon.

75. The method of claim 55, wherein the glass vial comprises a glass ampoule.

76. The method of claim 43, wherein the solid form quercetin composition comprises about 8:1, about 9:1, about 10:1, about 11:1 or about 12:1 polyvinylpyrrolidone:quercetin w/w.

77. The method of claim 43, further comprising determining the level of the toxic contaminant.

78. The method of claim 77, wherein the toxic contaminant is 2-(3,4-dihydroxybenzoyloxy)-4,6-dihydroxybenzoic acid.

79. The method of claim 77, wherein the determining step is performed at least three months after filling the container with an atmosphere consisting essentially of an inert gas or a combination of inert gases.

80. The method of claim 79, wherein the container is stored at 20-25° C.

81. The method of claim 77, wherein the determining step is performed at least six months after filling the container with an atmosphere consisting essentially of an inert gas or a combination of inert gases.

82. The method of claim 81, wherein the container is stored at 20-25° C.

83. The method of claim 77, wherein the determining step is performed at least twelve months after filling the container with an atmosphere consisting essentially of an inert gas or a combination of inert gases.

84. The method of claim 83, wherein the container is stored at 20-25° C.

85. The method of claim 77, wherein the determining step is performed at least twenty four months after filling the container with an atmosphere consisting essentially of an inert gas or a combination of inert gases.
